(19) United States
(12) Patent Application Publication
(10) Pub. No.: US 2003/0144216 A1
(43) Pub. Date: Jul. 31, 2003

(54) Method for prevention of diseases in coeliac patients

(76) Inventor: Mikko Unkila, Littoinen (FI)

Correspondence Address: James C. Lydon, Suite 100, 100 Daingerfield Road, Alexandria, VA, 22314, US

(21) Appl. No.: 10/054,900

(22) Filed: Jan. 25, 2002

(51) Int. Cl. A61K 31/70; A61K 35/78

(52) U.S. Cl. 514/22; 514/25; 424/769

(57) ABSTRACT

Methods for prevention of cancers, precancers, certain non-cancer, hormone dependent diseases and/or cardiovascular diseases in a person suffering from coeliac disease, based on administering of a lignan to the person. A method for increasing the level of enterolactone or another metabolite of a lignan in a person's serum is also disclosed, where the person suffers from coeliac disease, thereby causing prevention of a cancer or a certain non-cancer, hormone dependent disease in the person, based on administering of a lignan to the person.

### FIELD OF THE INVENTION

This invention relates to a method for prevention of a cancer, precancer, a certain non-cancer, hormone dependent disease and/or a cardiovascular disease in a person suffering from coeliac disease, comprising administering to said person an effective amount of a lignan. Particularly, the invention relates to prevention of coeliac-related diseases, such as coeliac-related cancers, in coeliac patients.

### BACKGROUND OF THE INVENTION

The publications and other materials used herein to illuminate the background of the invention, and in particular, cases to provide additional details respecting the practice, are incorporated by reference.

Coeliac disease, also known as gluten intolerance, is a genetic disorder that affects between 1 in 150 to 1 in 250 Americans (1, 2). Symptoms of coeliac disease can range from the classic features, such as diarrhea, weight loss, and malnutrition, to latent symptoms such as isolated nutrient deficiencies but no gastrointestinal symptoms. In coeliac patients, specific food-grain antigens (mainly gluten) present in wheat, rye and barley, damages the villi (shortening and villous flattening) in the lamina propria and crypt regions of their intestines.

The only acceptable treatment for coeliac disease is strict adherence to a 100% gluten-free diet for life. An adherence to a gluten-free diet can prevent almost all complications caused by the disease (3). A gluten-free diet means avoiding all products that contain wheat, rye and barley, or any of their derivatives.

Coeliac disease patients have been described to have considerably elevated risk of malignant cancer, especially that of small intestinal lymphoma (4,5,6). Also considerable higher risk of small intestinal adenocarcinoma and esophageal cancer (6) has been described. It has been estimated that up to 15% of patients with untreated or refractory coeliac disease develop these cancers (3). Other types of cancer that may have an elevated risk include cancers in the gastrointestinal tract and testicular cancer (5).

Lignans are defined as a class of phenolic compounds possessing a 2,3-dibenzylbutane skeleton. They are formed by coupling of monomeric units called precursors such as cinnamic acid, caffeic, ferulic, coumaric, and gallic acids (7). Lignans are widely distributed in plants. They can be found in different parts (roots, leafs, stem, seeds, fruits) but mainly in small amounts. In many sources (seeds, fruits) lignans are found as glycosidic conjugates associated with fiber component of plants. The most common dietary sources of mammalian lignan precursors are unrefined grain products. The highest concentrations in edible plants have been found in flaxseed, followed by unrefined grain products, particularly rye.

Considerable amounts of lignans are also found in coniferous trees. The type of lignans differs in different species and the amounts of lignans vary in different parts of the trees. The typical lignans in heart wood of spruce (Picea abies) are hydroxymatairesinol (HMR), α-conidendrin, conidendrinic acid, matairesinol, isolariciresinol, secoisolariciresinol, liovile, picearesinol, lariciresinol and pinoresinol (8). The far most abundant single component of lignans in spruce is HMR, about 60 percent of total lignans, which occurs mainly in unconjugated free form.

Plant lignans such as hydroxymatairesinol, matairesinol and secoisolariciresinol, are converted by gut microflora to mammalian lignans, enterolactone or enterodiol (9; WO 00/59946). A recent study (10) shows also that matairesinol, secoisolariciresinol, lariciresinol and pinoresinol glucoside were to be converted to enterolactone.

Enterolactone is known to possess many valuable therapeutical properties. Urinary excretion and serum concentrations of enterolactone are low in women diagnosed with breast cancer (11, 12) suggesting that this lignan is chemopreventive. The inhibition of aromatase by enterolactone would suggest a mechanism by which consumption of lignan-rich plant food might contribute to reduction of estrogen-dependent diseases, such as breast cancer (13, 14). The potential antioxidant activity of enterolactone could also represent a mechanism associated with the preventive action of this lignan in the development of cancers.

Methods for the synthesis of enterolactone has been disclosed in the literature (15). However, isolated mammalian lignans such as enterolactone, have not so far been available in sufficient amounts to be used in animal experiments or clinical trials. The only possibility to increase mammalian lignan supply has been to increase the consumption of fiber-rich food items such as flaxseed.

The international patent publication WO 00/59946 discloses that hydroxymatairesinol is efficiently converted to enterolactone in vivo and thus useful to increase the level of enterolcatone. The publication also indicates that hydroxymatairesinol can be effective as such due to its antioxidative activity in vitro. This publication discloses usefulness of hydroxymatairesinol in the prevention of cancers such as breast cancer, prostate cancer and colon cancer, non-cancer, hormonal dependent diseases such as lower urinary tract symptoms, urethral dyssynergia, bladder instability, bladder outlet obstruction, benign prostatic hyperplasia, and gynecomastia in men, and cardiovascular diseases resulting from oxidized LDL in serum.

The international patent publication WO 01/78720 suggests the use of hydroxymatairesinol to decrease the intracellular level of â-catenin and thereby prevent a precancerous condition, namely familial adenomatous polyposis (FAP).

### SUMMARY OF THE INVENTION

According to one aspect, this invention concerns a method for prevention of a cancer, precancer, a certain non-cancer, hormone dependent disease and/or a cardiovascular disease in a person suffering from coeliac disease, comprising administering to said person an effective amount of a lignan.

According to another aspect, the invention concerns a method for increasing the level of enterolactone or another metabolite of a lignan in a person's serum, wherein said patient suffers from coeliac disease, thereby causing prevention of a cancer or a certain non-cancer, hormone dependent disease in said person, comprising administering to said person an effective amount of a plant lignan.

### DETAILED DESCRIPTION OF THE INVENTION

Whole grain cereal crop products, especially those made from rye, contain significant amounts of lignans and thereby they constitute a significant dietary source of mammalian lignan precursors, such as enterolactone precursors in the human diet. Since coeliac patients must avoid grain products, they have consistently a decreased supply of mammalian lignan precursors, such as enterolactone precursors, in their diet. Thus, it is likely that coeliac patients may have lower levels of blood enterolactone and other mammalian lignans, compared with average population.

Persons with coeliac disease can typically compensate their carbohydrate intake for example by increasing proportional intake of soy and potato. Also coeliac people are able to ingest oat in their daily diet. Importantly, when compared to for example rye, the levels of lignans (SECO and MAT) are considerably less in these dietary sources (16). Lignan concentrations in various foods can be seen in Table 1.

TABLE 1
Lignan concentrations (ig/100 dry weight) in various foods

| | SECO | MAT | |
|---|---|---|---|
| Soybean | 13-273 | (variable) | trace |
| Potato | 10 | | 6 |
| Oat meal | 13 | 0 | |
| Rye bran | 132 | 167 | |
| Rye meal | 47 | 65 | |

SECO = secoisolariciresinol,
MAT = matairesinol

Furthermore, it was recently shown that consumption of rye bread constitutes a significant source of bioavailable lignan precursors elevating blood enterolactone levels (17). Thus, it may be concluded that coeliac disease patients who cannot consume enough lignan rich grain products likely have a lignan deficit in their diet.

The term “coeliac disease” shall in the definition of the present invention be understood to include glutene intolerance and any other intolerance to grain products. Accordingly, a “coeliac patient” shall be understood as a patient suffering from a disease so defined.

The diseases which can be prevented by the method according to this invention are, for example, cancers such as small intestinal cancers, colon cancer, breast cancer, prostate cancer, cancer in the gastrointestinal tract and testicular cancer; precancers such as familial adenomatous polyposis (FAP); non-cancer, hormonal dependent diseases such as lower urinary tract symptoms, urethral dyssynergia, bladder instability, bladder outlet obstruction, benign prostatic hyperplasia, and gynecomastia in men; and cardiovascular diseases resulted from oxidized LDL in serum.

Although the method according to the present invention can be used to prevent diseases generally correlating with a lignan-deficiency in a patient suffering from coeliac disease, the method is particularly valuable for prevention of diseases which additionally are shown to be coeliac-related. The current evidence suggests an elevated risk of certain cancer in coeliac disease patients. Such cancers are, for example, small intestinal lymphoma, small intestinal adenocarcinoma, esophageal cancer, cancers in the gastrointestinal tract or testicular cancer. Therefore it is particularly preferable to administer lignans to coeliac patients to reduce the risk of these cancers.

As a first alternative, a decreased level of mammalian lignans, especially enterolactone, in blood appears as a risk predictor of certain cancer (breast/prostate), but may also act to modulate the risk of other cancer. Therefore, promoting the lignan precursor poor diet of coeliac patients with a plant lignan may be effective in elevating the lower levels of mammalian lignans such as enterolactone in coeliac patients. By elevating the blood enterolactone concentration to suitable level (e.g. 50-200 nMol/l), can have a cancer chemopreventive activity.

As a second alternative, since lignans have several putative beneficial properties as nutritional supplements (e.g. they are antioxidants), coeliac patients may also benefit from direct effects associated with plant lignans. Therefore, addition of a plant lignan in daily diet may elicit beneficial health effects without conversion to mammalian lignans such as enterolactone. This assumption can be based of the facts that 1) coeliac patients may have a chronically inflamed intestinal mucosa, which in persistent situation may lead to the development of cancer, and 2) plant lignans such as hydroxymatairesinol, are strong antioxidants and have mild suppressive effect on overactive inflammatory cells. Thus, by inhibiting the inflammation, the plant lignan can inhibit development of precancerous and cancerous lesions.

As a third alternative, the mammalian lignan, e.g. enterolactone or enterodiol, can be administered as such to the coeliac patient.

Preferred plant lignans are, for example, hydroxymatairesinol, allohydroxymatairesinol, matairesinol, lariciresinol, secoisolariciresinol, isolariciresinol, oxomatairesinol, conidendrin, conidendric acid, pinoresinol, pinoresinol glucoside, liovil, picearesinol, nortrachelogenin, arctigenin, and their geometric isomers and stereoisomers, salts and adducts, and mixtures.

Particularly preferred are the plant lignans hydroxymatairesinol, matairesinol, lariciresinol, secoisolariciresinol, pinoresinol and pinoresinol glucoside, and their geometric isomers and stereoisomers, salts and adducts, and mixtures. These lignans have been shown a good ability to be converted into enterolactone.

Preferred mammalian lignans are enterolactone and enterodiol, especially enterolactone.

The lignan to be administered to the coeliac patient shall in this text be understood to cover any geometric isomer or stereoisomer or any mixture of isomers, such as racemates, of these compounds. Salts, adducts and complexes of the compounds shall also be understood to be covered by the term.

The lignans to be used in this invention can be supplied in the form of a pharmaceutical preparation, dietary supplement, clinical nutrition formula or as a functional food. According to a particularly preferred embodiment, the lignan is administered as a dietary supplement for clinical nutritional purposes to the coeliac patients.

The pharmaceutical preparation according to this invention is preferably an oral formulation. The required amount of the active compound or mixture of compounds will vary with the compound and the particular condition to be prevented. A typical dose ranges from about 10 to about 2000 mg per day and adult person, preferably 100 to 600 mg per day and adult person. Typical dosage forms include, but are not limited to, oral dosage forms such as powders, granules, capsules, tablets, caplets, lozenges, liquids, elixirs, emulsions and suspensions. All such dosage forms may include conventional carriers, diluents, excipients, binders and additives known to those skilled in the medicinal and pharmaceutical arts.

The pharmaceutical or other formula carriers typically employed may be solid or liquid. Thus, for example, solid carriers include polysaccarides such as lactose, sucrose, gelatin, agar, while liquid carriers include aqueous solutions of salts, polysaccarides, complexing agents, surfactants, syrups, vegetable oils such as peanut oil or olive oil, and certain alcohols. However, any acceptable solid or liquid carrier can be used in the pharmaceutical preparation or other dietary or nutrition formula to be administered according to this invention.

A typical food product, suitable for use in the methods according to this invention, is especially a functional food, a nutritional supplement, a nutrient, a pharmafood, a nutraceutical, a health food, a designer food or any food product. The term food product shall also be understood to cover groceries and foodstuffs such as flour, other ingredients, certain liquids, etc. A suitable concentration of the active compound in the food product is, for example, 5 to 1000 mg of active compound per 100 g of food product, preferably about 10 to 100 mg of active compound per 100 g of food product.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

### REFERENCES

1. University of Maryland Center for Celiac Research, Multi-Center Serological Screening Study Results, Alessio Fasano, M.D., Karoly Horvath M.D./Ph.D., May 20, 2000 (http://celiaccenter.org/frm_research_update.htm)

2. Gastroenterology, April, 1996 “First Epidemiological Study of Gluten Intolerance in the United States.” By Karoly Horvath, M.D., Ph.D., et al.

3. New England Journal of Medicine, May 2, 1996—Volume 334, Number 18, “The Many Faces of Celiac Disease” by Charles H. Halsted, M.D.

4. Cooper B T, Holmes G K T, Ferguson R. and Cooke W T (1980) Celiac disease and malignancy. Medicine 59, 249-261

5. Swinson C, Slavin G, Coles E C and Bootj C C. (1983) Coeliac disease and malignancy. Lancet January 15, 111-115.

6. Ferguson S and Kingstone K (1996) Coeliac disease and malignancies. Acta Paediatr Suppl. 412, 78-81.

7. Ayres D, and Loike, J. Lignans: Chemical, biological and clinical properties. Cambridge university press, 1990.

8. Elman R: Distribution of lignans in Norway spruce. Acta Academiae Aboensis, Ser B, 39:1-6, 1979.

9. Axelson M, Sjövall J, Gustafsson B E and Setchell K D R: Origin of lignans in mammals and identification of a precursor from plants. Nature, 298: 659-660, 1982.

10. Heinonen S, Nurmi T, Liukkonen K, Poutanen K, Wahala K, Deyama T, Nishibe S, Adlercreutz H (2001) In vitro metabolism of plant lignans: new precursors of mammalian lignans enterolactone and enterodiol. J Agric Food Chem, 49, 3178-86.

11. Ingram D, Sanders K. Kolybaba M and Lopez D. Case-control study of phyto-oestrogens and breast cancer. Lancet, October 4;350(9083) 990-994, 1997.

12. Hultén K, Adlercreutz H, Winkvist A, Lenner P, Hallmans G and Ågren Å. Low levels of phyto-estrogens in blood as risk factor for breast cancer. In: COST 916 Workshop ‘Phyto-oestrogens: exposure, bioavailability, health benefits and safety concerns’, 1998

13. Adlercreutz H, Bannwart C, Wähälä K, Mäkelä T, Brunow G, Hase T, Arosemena P J, Kellis J T, and Vickery L E: Inhibition of human aromatase by mammalian lignans and isoflavonoid phytoestrogens. J Steroid Biochem Mol Biol, 44: 147-153, 1993.

14. Wang C, Mäkelä T, Hase T, Adlercreutz H and Kurzer M S: Lignans and flavonoids inhibit aromatase enzyme in human adipocytes. J Steroid Biochem Molec Biol, 50: 205-212, 1994.

15. M B Groen and J Leemhuis, Tetrahedron Letters 21, 5043 (1980) and G Cooley et al., ibid 22, 349 (1981).

16. Mazur and Adlercreutz (1998) Natural and anthropogenic environmental oestrogens: the scientific basis for risk assessment. Naturally occurring oestrogens in food. Pure Appl. Chem. 70, 1759-1776.

17. Juntunen K S, Mazur W M, Liukkonen K H, Uehara M, Poutanen K S, Adlercreutz H C, Mykkanen H M (2000): Consumption of wholemeal rye bread increases serum concentrations and urinary excretion of enterolactone compared with consumption of white wheat bread in healthy Finnish men and women. Br. J. Nutr. 84: 839-846.

1. A method for prevention of a cancer, precancer, a certain non-cancer, hormone dependent disease and/or a cardiovascular disease in a person suffering from coeliac disease, comprising administering to said person an effective amount of a lignan.

2. The method according to claim 1 wherein said cancer or precancer is selected from the group consisting of small intestinal cancers, colon cancer, familial adenomatous polyposis (FAP), breast cancer, prostate cancer, cancer in the gastrointestinal tract and testicular cancer.

3. The method according to claim 1 wherein the disease is a coeliac-related disease.

4. The method according to claim 3 wherein the disease is small intestinal lymphoma, small intestinal adenocarcinoma, esophageal cancer, cancers in the gastrointestinal tract or testicular cancer.

5. The method according to claim 1 wherein said non-cancer, hormonal dependent disease is selected from the group consisting of lower urinary tract symptoms, urethral dyssynergia, bladder instability, bladder outlet obstruction, benign prostatic hyperplasia, and gynecomastia in men.

6. The method according to claim 1 wherein said cardiovascular disease is resulted from oxidized LDL in serum.

7. The method according to claim 1 wherein the lignan is a plant lignan.

8. The method according to claim 7 wherein the plant lignan is selected from the group consisting of hydroxymatairesinol, allohydroxymatairesinol, matairesinol, lariciresinol, secoisolariciresinol, isolariciresinol, oxomatairesinol, conidendrin, conidendric acid, pinoresinol, pinoresinol glucoside, liovil, picearesinol, nortrachelogenin, arctigenin, their geometric isomers and stereoisomers, salts and adducts, and mixtures thereof.

9. The method according to claim 8 wherein the plant lignan is selected from a group consisting of hydroxymatairesinol, matairesinol, lariciresinol, secoisolariciresinol, pinoresinol and pinoresinol glucoside, their geometric isomers and stereoisomers, salts and adducts, and mixtures thereof.

10. The method according to claim 1 wherein the lignan is a mammalian lignan.

11. The method according to claim 10 wherein the mammalian lignan is selected from a group consisting of enterolactone and enterodiol and their geometric isomers and stereoisomers, and mixtures thereof.

12. A method for increasing the level of enterolactone or another metabolite of a lignan in a person's serum, wherein said patient suffers from coeliac disease, thereby causing prevention of a cancer or a certain non-cancer, hormone dependent disease in said person, comprising administering to said person an effective amount of a plant lignan.

13. The method according to claim 12 wherein said cancer or precancer is selected from the group consisting of small intestinal cancers, colon cancer, familial adenomatous polyposis (FAP), breast cancer, prostate cancer, cancer in the gastrointestinal tract and testicular cancer.

14. The method according to claim 12 wherein the disease is a coeliac-related disease.

15. The method according to claim 14 wherein the disease is small intestinal lymphoma, small intestinal adenocarcinoma, esophageal cancer, cancers in the gastrointestinal tract or testicular cancer.

16. The method according to claim 12 wherein said non-cancer, hormonal dependent disease is selected from the group consisting of lower urinary tract symptoms, urethral dyssynergia, benign prostatic hyperplasia, and gynecomastia in men.

17. The method according to claim 12 wherein the plant lignan is selected from the group consisting of hydroxymatairesinol, allohydroxymatairesinol, matairesinol, lariciresinol, secoisolariciresinol, isolariciresinol, oxomatairesinol, conidendrin, conidendric acid, pinoresinol, pinoresinol glucoside, liovil, picearesinol, nortrachelogenin, arctigenin, their geometric isomers and stereoisomers, salts and adducts, and mixtures thereof.

18. The method according to claim 17 wherein the plant lignan is selected from a group consisting of hydroxymatairesinol, matairesinol, lariciresinol, secoisolariciresinol, pinoresinol and pinoresinol glucoside, their geometric isomers and stereoisomers, salts and adducts, and mixtures thereof.
